(19) United States
(12) Patent Application Publication
(10) Pub. No.: US 2025/0001119 A1
(43) Pub. Date: Jan. 2, 2025

(54) VARIABLE RESISTANCE HEAT AND MOISTURE EXCHANGER

(71) Applicant: KONINKLIJKE PHILIPS N.V., EINDHOVEN (NL)

(72) Inventors: THEODOOR STOLK, LANGEZWAAG (DE); BERNARD F. HETE, KITTANNING, PA (US); HENDRIK KLAAS LOUWSMA, DRACHTSTERCOMPAGNIE (NL); KLAAS KOOIJKER, DRACHTEN (DE)

(21) Appl. No.: 18/756,214

(22) Filed: Jun. 27, 2024

Related U.S. Application Data
(60) Provisional application No. 63/523,753, filed on Jun. 28, 2023.

(51) Int. Cl. A61M 16/10; A61M 16/06; A61M 16/20

(52) U.S. Cl. CPC A61M 16/1045; A61M 16/0605; A61M 16/20

(57) ABSTRACT

An HME for use in providing a flow of breathing gas to a patient includes a housing defining a main passage for conveying the flow of breathing gas therethrough as well as an HME element and a number of valve arrangements positioned within the main passage. Each valve arrangement includes a valve passage and a valve member moveable among a first position wherein the passage of gases through the valve passage is prevented and thus flow in the main passage is required to pass through the HME element, and a second position in which the passage of gases through the valve passage is permitted and thus flow in the main passage can bypass the HME element. The positioning of the valve member of each valve arrangement among the first position and the second position is dependent on a flow of gas through the main passage.

### CROSS-REFERENCE TO RELATED APPLICATIONS

This patent application claims the priority benefit under 35 U.S.C. § 119(e) of U.S. Provisional Application No. 63/523,753, filed on Jun. 28, 2023, the contents of which are herein incorporated by reference.

### BACKGROUND OF THE INVENTION

### 1. Field of the Invention

The disclosed concept relates generally to heat and moisture exchangers for use in arrangements providing a flow of breathing gas to a patient and, more particularly, to heat and moisture exchangers that present a variable resistance to gases passing therethrough.

### 2. Description of the Related Art

Breathing apparatus are used to provide breathing assistance to patients. Examples of such breathing apparatus are CPAP (continuous positive airway pressure), bi-level and/or autotitration PAP (positive airway pressure) apparatus that provide pressure support to a patient for treating obstructive sleep apnea or other breathing disorders. Other examples of such breathing apparatus are ventilators (such as non-invasive ventilation-NIV, as well as invasive ventilation) that provide assisted breathing or flow therapy.

Often as part of therapies provided by such devices and/or for comfort to the patient it is desirable to humidify the air provided to the patient by the breathing apparatus. Such humidity may be provided actively, e.g., via humidifiers, or passively, e.g., via a heat and moisture exchanger (HME), or a combination thereof. HME's function to scavenge heat and humidity from gases exhaled by a patient in order to provide heat and humidity to new gases being provided to the patient. Presently, HME's are a consumable product-after one (sometimes multiple) day(s) of use the component is disposed and replaced by a new one. In such HME's, the active element (i.e., the element scavenging/transferring heat/humidity from exhaled gases to new gases being provided to the patient) is typically a sponge like material or rolled paper arrangement.

The goal of an HME is to capture the heat and moisture from the patients exhaled air. This heat and moisture are released again upon inhaling the cooler and relatively dry ambient air. Through such device the air inhaled by the patient is humidified and heated by his/her exhaled moisture and heat, respectively. HME's are typically, and preferably simple low-cost devices. One of the primary disadvantages with HME's is that they increase patient circuit deadspace due to the fact that they must reside between the patient and the exhalation valve. Unfortunately, the best approach to reduce deadspace is to reduce volume of the HME, but this normally also results in a concomitant reduction in recovery material surface area, which then reduces thermal recovery. Further, HMEs tend to increase flow resistance with duration of use, and peer-reviewed publications have been concerned over the possibility of their total occlusion, even though this extreme event is not well documented.

### SUMMARY OF THE INVENTION

Embodiments of the present invention improve upon existing solutions by providing HME arrangements that utilize internal valving to increase the effectiveness of the HME element with minimal affect to complexity and cost. By increasing the effectiveness of the HME element, the HME element can be smaller because energy is only delivered at the end of exhalation when the flows are lower, and the pressure drop across the HME is lower.

As a first aspect of the present invention, an HME for use in providing a flow of positive pressure breathing gas to the airway of a patient is provided. The HME comprises: a housing defining a main passage therethrough, the main passage structured to convey the flow of positive pressure breathing gas therethrough; an HME element positioned within the main passage; and a number of valve arrangements positioned within the main passage, each valve arrangement having a valve passage and a valve member moveable among: a first position wherein the valve member is structured to prevent the passage of gases through the valve passage and thus require flow in the main passage to pass through the HME element, and a second position in which the valve member is structured to permit the passage of gases through the valve passage and thus permit flow in the main passage to bypass the HME element, wherein the positioning of the valve member of each valve arrangement among the first position and the second position is dependent on a flow of gas through the main passage.

The number of valve arrangements may be structured to provide for flow in the main passage to bypass the HME element during a portion of inspiration by the patient and to inhibit flow in the main passage from bypassing the HME element during expiration by the patient.

The HME element may be positioned centrally in the main passage.

The number of valve arrangements may comprise a plurality of valve arrangements.

The number of valve arrangements may comprise a plurality of valve arrangements spaced circumferentially within the main passage about the HME element.

Each valve arrangement may comprise a flapper valve.

Each valve arrangement may comprise a biasing member structured to bias the valve arrangement toward a closed position.

As another aspect of the present invention a respiratory interface system for use in providing a regimen of respiratory therapy to a patient is provided. The respiratory interface system comprises: a pressure generating device structured to generate a flow of positive pressure breathing gas; a patient interface structured to engage an airway of the patient; a delivery conduit arrangement having a first end coupled to the pressure generating device and an opposite second end coupled to the patient interface, the delivery conduit arrangement structured to communicate the flow of positive pressure breathing gas from the pressure generating device to the patient interface such that the delivery conduit arrangement and patient interface define a flow path which is structured to convey the flow of positive pressure breathing gas from the pressure generating device to the patent; and an HME positioned along the flow path, the HME comprising: a housing defining a main passage therethrough, the main passage structured to convey the flow of positive pressure breathing gas; an HME element positioned within the main passage; and a number of valve arrangements positioned within the main passage, wherein each valve arrangement of the number of valve arrangements having a valve passage and a valve member moveable among: a first position wherein the valve member is structured to prevent the passage of gases through the valve passage and thus require flow in the main passage to pass through the HME element, and a second position in which the valve member is structured to permit the passage of gases through the valve passage and thus permit flow in the main passage to bypass the HME element, and wherein the positioning of the valve member of each valve arrangement among the first position and the second position is dependent on a flow of gas through the main passage.

The number of valve arrangements may be structured to provide for flow in the main passage to bypass the HME element during a portion of inspiration by the patient and to inhibit flow in the main passage from bypassing the HME element during expiration by the patient.

The HME element may be positioned centrally in the main passage.

The number of valve arrangements may comprise a plurality of valve arrangements.

The number of valve arrangements may comprise a plurality of valve arrangements spaced circumferentially within the main passage about the HME element.

Each valve arrangement may comprise a flapper valve.

Each valve arrangement may comprise a biasing member structured to bias the valve arrangement toward a closed position.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

A full understanding of the invention can be gained from the following description of the preferred embodiments when read in conjunction with the accompanying drawings in which:

FIG. 1 is a respiratory interface system including an HME in accordance with an example embodiment of the present invention;

FIG. 2 is a schematic axial elevation view of the HME of FIG. 1 as viewed along a flowpath therethrough;

FIG. 3 is schematic sectional view of the HME of FIGS. 1 and 2 taken along 3-3 of FIG. 1 showing the HME during an expiration phase of breathing by a patient;

FIG. 4 is another schematic sectional view of the HME of FIGS. 1 and 2 similar to the view of FIG. 3 except showing the HME during an initial, high flow, portion of an inspiration phase of breathing by a patient; and

FIG. 5 is yet a further schematic sectional view of the HME of FIGS. 1 and 2 similar to the view of FIG. 3 except showing the HME during a later, low flow, portion of an inspiration phase of breathing by a patient.

### DETAILED DESCRIPTION OF THE INVENTION

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As used herein, the statement that two or more parts or components “engage” one another shall means that the parts exert a force against one another either directly (i.e., “directly engage”) or through one or more intermediate parts or components. As used herein, the word “unitary” means a component is created as a single piece or unit. That is, a component that includes pieces that are created separately and then coupled together as a unit is not a “unitary” component or body. As used herein, the term “number” shall mean one or an integer greater than one (i.e., a plurality).

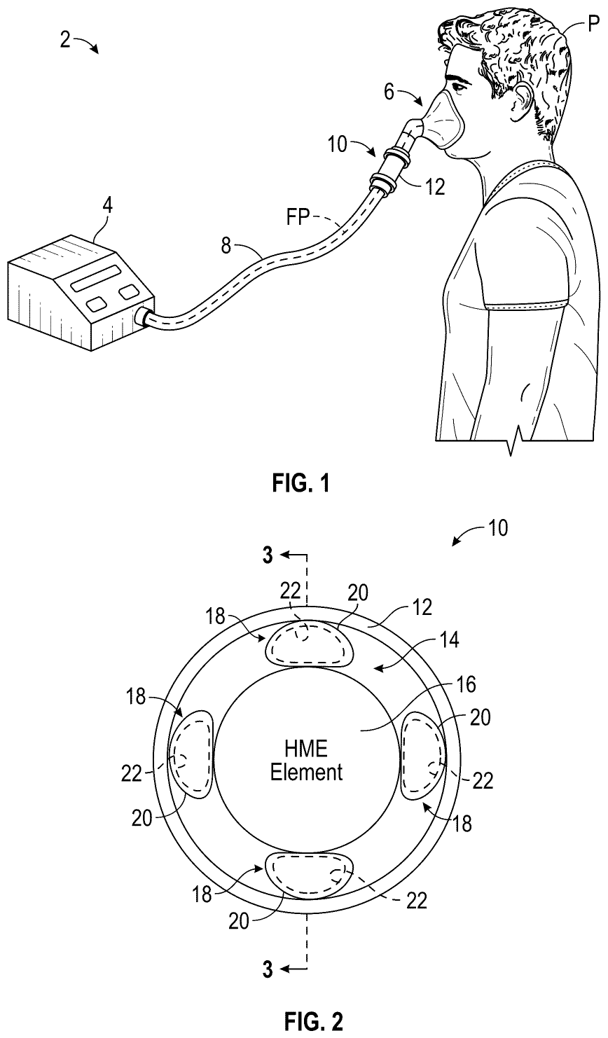

FIG. 1 shows a respiratory interface system 2 for use in providing a regimen of respiratory therapy to a patient P according to one particular, non-limiting example embodiment of the present invention. Respiratory interface system 2 includes a pressure generating device 4, a patient interface 6, and a delivery conduit arrangement 8. Delivery conduit arrangement 8 includes a first end and an opposite second end, with the first end coupled to pressure generating device 4 and the opposite second end coupled to patient interface 6. Pressure generating device 4 is structured to generate a flow of positive pressure breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, PA), and auto-titration pressure support devices. Delivery conduit arrangement 8 may be formed from one or more (i.e., a number of) conduit sections/couplings (not numbered) and is structured to communicate the flow of positive pressure breathing gas from pressure generating device 4 to patient interface 6. Hence, delivery conduit arrangement 8 and patient interface 6 define a flow path FP (shown in dashed line) from pressure generating device 4 to patient P.

Continuing to refer to FIG. 1 and additionally to FIGS. 2-5, respiratory interface system 2 further includes a heat and moisture exchanger (HME) 10 positioned along flow path FP. In the example shown in FIG. 1, HME 10 is positioned immediately adjacent patient interface 6, however, it is to be appreciated that HME 10 may be positioned at other locations along flow path FP (e.g., without limitation, spaced from patient interface 6, provided as a portion of patient interface 6) without varying from the scope of the present invention. HME 10 includes a housing 12 defining a passage 14 therethrough. Housing 12 may be formed from one or more materials the same as/similar to conduit arrangement 8 and/or any other suitable material(s). When HME 10 is positioned within respiratory interface system 2, passage 14 defines a portion of flow path F.

As shown schematically in FIGS. 2-5, HME 10 further includes an HME element 16 and a number of valve arrangements 18, positioned within passage 14 such that any gases passing through passage 14 must pass through either HME element 16 or one of the number of valve arrangements 18. HME element 16 may be of any suitable construction/design for capturing heat/moisture from exhaled gases passing therethrough from a patient and transferring such heat/moisture to the flow of positive pressure breathing gas provided by pressure generating device 4 prior to being inhaled by the patient. As arrangements of HME elements are commonly known to those of ordinary skill in the art, further description of HME element 16 is not provided herein. Each valve arrangement 18 may be any suitable valve arrangement for use in allowing, limiting, and preventing a flow of gas therethrough based solely on the flow of gas and/or pressure resulting therefrom in passage 14 such as described further below. In the example shown schematically in FIGS. 2-5, each valve arrangement 18 is a flapper valve arrangement including a flapper member 20 and a valve passage 22. As is known, in such a flapper valve arrangement flapper member 20 is moveable among an open position in which gas may pass through valve passage 22 and a closed position in which gas is prevented from passing through valve passage 22 by flapper member 20.

Flapper member 20 may be biased in the closed position by internal forces (e.g., via stiffness of the flap material) and/or via an external arrangement (e.g., a spring). In the example shown in FIGS. 2-5, the number of valve arrangements 18 comprises a plurality of valve arrangements 18 (e.g., without limitation four valve arrangements 18 are shown in such example) spaced circumferentially within passage 14 about HME element 16 (which is disposed centrally in passage 14). It is to be appreciated, however, that one or more of the quantity and/or positioning of the number of valve arrangements 18 and/or HME element 16 may be varied without varying from the scope of the present invention.

In the example embodiment shown in FIGS. 2-5, as well as other example embodiments of the present invention, one or more valve arrangements 18 are employed to allow at least some gas to bypass the HME energy storage material (i.e., HME element 16) during high flows when pressure drop across HME element 16 is greatest, then require all flow to pass through HME element 16 when the flow speed is lower (and pressure drop is negligible). Such arrangement allows the humidity to be recovered ideally during the end of the inspiratory phase of the patient so that more water is delivered to air that will fill anatomic deadspace of the patient, the portion of the patient airway most in need of humidification. The idea behind such approach is that it is desired to provide the most humidity at the end of the inspiratory phase of a patient so that the upper airways can be wetted. Early phase inspiration gas goes into the lower airways of the patient, portions that are already highly wetted. The problem with a conventional HME upon which embodiments of the present improve is that the air gets drier toward the end of inspiration because the water starts to leave the HME energy storage material as soon as flow passes through the HME, and the water is thus delivered primarily to the lower airways of the patient, i.e., portions of the airways that are in less need of moisture.

The approach described herein saves the humid air for the upper airways of the patient that are filled at the end of inspiration. The physiologic benefit of such approach is that the upper airways are the site of the mucociliary escalator, the function of which is to move foreign matter out of the airway. This function is often compromised in chronic ventilator patients and one key to helping maintain such function is to maintain the wetness of this portion of the airway of the patient.

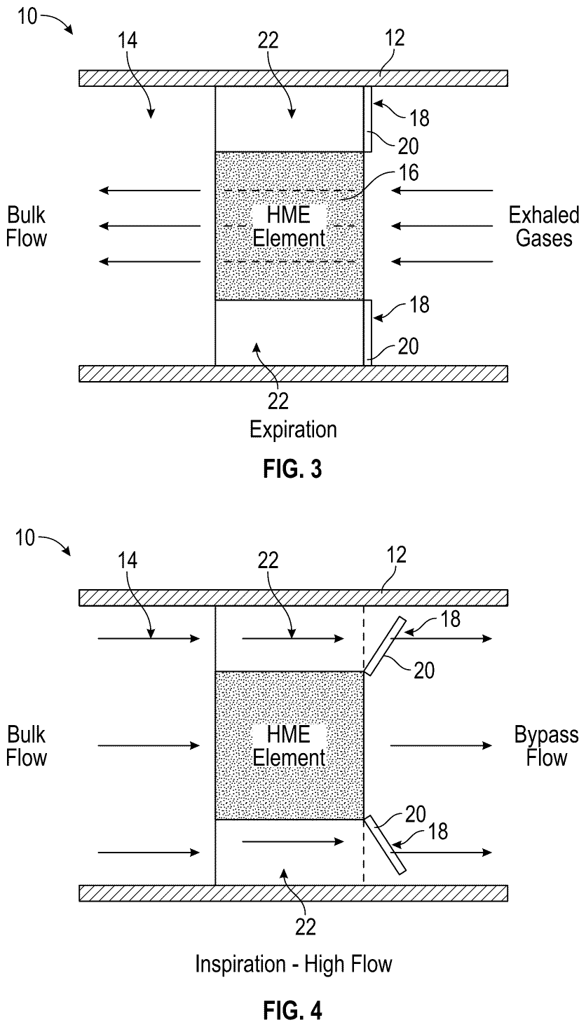

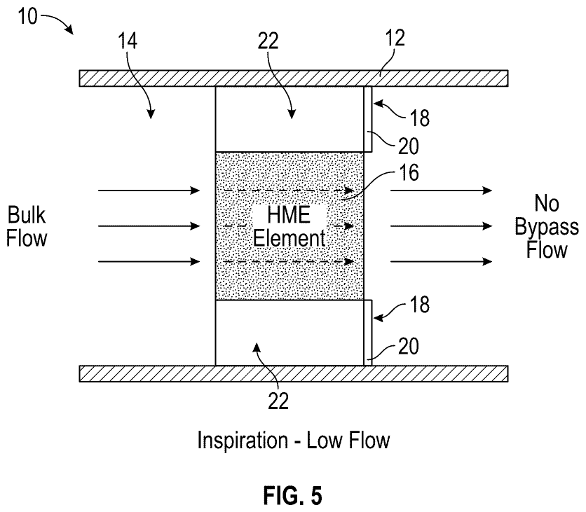

General functionality of HME 10 will now be described in conjunction with the schematic views of FIGS. 3-5. Starting with FIG. 3, flow through passage 14 of HME 10 during the expiratory phase of a patient is shown. During expiration, the pressure drop across HME element 16 (due to the flow resistance of HME element 16) acts to close flapper member 20 of each valve arrangement 18, thus preventing bypass flow around HME element 16 through the associated valve passage 22, which forces all exhaled flow from the patient through HME element 16, thus charging HME element 16 for the next inhale by the patient. Moving now to FIG. 4, flow through passage 14 of HME 10 during the initial, high flow portion, of the inspiratory phase of a patient is illustrated. During such phase, the high flow toward the patient causes flapper member 20 of each valve arrangement 18 to open and deliver flow around HME element 16 (i.e., the path of least resistance) such that less water is picked up by the flow and removed from HME element 16. In the lower flow portion of the latter inspiratory phase, such as shown in FIG. 5, flapper member 20 of each valve closes under its own internal stiffness (and/or via assistance from a biasing member, e.g., without limitation, a spring), which prevents flow bypass and forces all flow through HME element 16. This function preserves the higher humidity air to be delivered to the upper airway since it is filled last during inspiration.

From the foregoing it is thus to be appreciated that the utility described herein could be employed to either direct more humidity to the end of inspiration in a normally sized HME, or to decrease the size of the HME to reduce deadspace.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of disclosed concept which is to be given the full breadth of the claims appended and any and all equivalents thereof.

What is claimed is:

1. An HME for use in providing a flow of positive pressure breathing gas to the airway of a patient (P), the HME comprising:
a housing defining a main passage therethrough, the main passage structured to convey the flow of positive pressure breathing gas therethrough;
an HME element positioned within the main passage; and
a number of valve arrangements positioned within the main passage, each valve arrangement having a valve passage and a valve member moveable among:
a first position wherein the valve member is structured to prevent the passage of gases through the valve passage and thus require flow in the main passage to pass through the HME element, and
a second position in which the valve member is structured to permit the passage of gases through the valve passage and thus permit flow in the main passage to bypass the HME element,
wherein the positioning of the valve member of each valve arrangement among the first position and the second position is dependent on a flow of gas through the main passage.

2. The HME of claim 1, wherein the number of valve arrangements are structured to provide for flow in the main passage to bypass the HME element during a portion of inspiration by the patient and to inhibit flow in the main passage from bypassing the HME element during expiration by the patient.

3. The HME of claim 1, wherein the HME element is positioned centrally in the main passage.

4. The HME of claim 1, wherein the number of valve arrangements comprises one valve arrangement or a plurality of valve arrangements.

5. The HME of claim 3, wherein the number of valve arrangements comprises a plurality of valve arrangements spaced circumferentially within the main passage about the HME element.

6. The HME of claim 1, wherein each valve arrangement comprises a flapper valve.

7. The HME of claim 1, wherein each valve arrangement comprises a biasing member structured to bias the valve arrangement toward a closed position.

8. A respiratory interface system for use in providing a regimen of respiratory therapy to a patient (P), the respiratory interface system comprising:
a pressure generating device structured to generate a flow of positive pressure breathing gas;
a patient interface structured to engage an airway of the patient;
a delivery conduit arrangement having a first end coupled to the pressure generating device and an opposite second end coupled to the patient interface, the delivery conduit arrangement structured to communicate the flow of positive pressure breathing gas from the pressure generating device to the patient interface such that the delivery conduit arrangement and patient interface define a flow path (FP) which is structured to convey the flow of positive pressure breathing gas from the pressure generating device to the patent; and
an HME positioned along the flow path, the HME comprising:
a housing defining a main passage therethrough, the main passage structured to convey the flow of positive pressure breathing gas;
an HME element positioned within the main passage; and
a number of valve arrangements positioned within the main passage,
wherein each valve arrangement of the number of valve arrangements having a valve passage and a valve member moveable among:
a first position wherein the valve member is structured to prevent the passage of gases through the valve passage and thus require flow in the main passage to pass through the HME element, and
a second position in which the valve member is structured to permit the passage of gases through the valve passage and thus permit flow in the main passage to bypass the HME element, and
wherein the positioning of the valve member of each valve arrangement among the first position and the second position is dependent on a flow of gas through the main passage.

9. The system of claim 8, wherein the number of valve arrangements are structured to provide for flow in the main passage to bypass the HME element during a portion of inspiration by the patient and to inhibit flow in the main passage from bypassing the HME element during expiration by the patient.

10. The system of claim 8, wherein the HME element is positioned centrally in the main passage.

11. The system of claim 8, wherein the number of valve arrangements comprises one valve arrangement or a plurality of valve arrangements.

12. The system of claim 10, wherein the number of valve arrangements comprises a plurality of valve arrangements spaced circumferentially within the main passage about the HME element.

13. The system of claim 8, wherein each valve arrangement comprises a flapper valve.

14. The system of claim 8, wherein each valve arrangement comprises a biasing member structured to bias the valve arrangement toward a closed position.
